(19) United States
(12) Patent Application Publication
(10) Pub. No.: US 2024/0005275 A1
(43) Pub. Date: Jan. 4, 2024

(54) SYSTEM AND USER INTERFACE FOR PRODUCING A RECIPE FOR CURABLE COMPOSITIONS

(71) Applicant: Betolar Oy, Kannonkoski (FI)

(72) Inventors: Juha Leppänen, Kannonkoski (FI); Olli-Pekka Kallasvuo, Helsinki (FI)

(21) Appl. No.: 18/253,746

(22) PCT Filed: Nov. 19, 2021
(86) PCT No.: PCT/FI2021/050789

§ 371 Date: May 19, 2023

(30) Foreign Application Priority Data
Nov. 20, 2020 (FI) 20206181

(51) Int. Cl. G06Q 10/0875; G06Q 10/0631; G06Q 50/04

(52) U.S. Cl. CPC G06Q 10/0875; G06Q 10/06315; G06Q 50/04; C04B 12/005

(57) ABSTRACT

The embodiments relate to a system, devices, methods, and computer programs for determining a recipe for curable compositions. The system may receive information related to available sidestream based and/or virgin raw materials suitable for production of curable products. In addition, the system may receive a request to deliver a recipe for a curable end-product. The request may include target feature information of an end-product. The system may further determine the recipe for the requested end-product on basis of the received target information and the information related to the available raw materials. In addition, the system may provide separate user interface and/or communication interfaces for raw material producers and end-product manufacturers.

### TECHNICAL FIELD

The present description relates to the production of curable compositions. Some of the disclosed embodiments relate to the use of sidestream based or virgin raw materials suitable for production of curable compositions.

### BACKGROUND

Various sidestreams are generated in industrial processes, the valorisation of which makes sense not only from an economic but also from an environmental point of view. One potential application for industrial sidestream based raw materials is the production of curable compositions for replacing concrete-based products.

### SUMMARY

This summary presents some simplified concepts, which will be described in more detail in the detailed description of the present description. This summary is not intended to define the key features or essential features of the application examples, nor is it intended to limit the embodiments set forth in the claims.

According to one embodiment, the system may comprise means for: receiving information related to available sidestream based and/or virgin raw materials suitable for production of curable products; receiving a request to deliver a recipe of a curable product or product component, the request comprising target feature information of the curable product or product component; and/or determining a recipe for producing the requested curable product or product component on basis of target feature information of the requested curable product or product component and information related to available sidestream based and/or virgin raw materials.

According to one embodiment, the system may further comprise: a first user interface and/or communication interface adapted to receive information related to available sidestream based and/or virgin raw materials suitable for production of curable products; and/or a second user interface and/or communication interface adapted to receive a request to deliver a recipe of a curable product or product component and to send the determined recipe for producing the requested curable product or product component.

According to one embodiment, at least some of the raw materials in the determined recipe may include sidestream based and/or virgin raw materials according to the information received via the first user interface and/or communication interface.

According to one embodiment, the system may further comprise means for: determining target feature information related to sidestream based raw materials suitable for production of curable products to improve usability of at least one sidestream based raw material in production of the curable product or product component on basis of the target feature information of the curable product or product component; and wherein the first user interface and/or communication interface is adapted to send target feature information related to sidestream based raw materials suitable for production of curable products.

According to one embodiment, the second user interface and/or communication interface may further be adapted to receive information on available raw materials of the manufacturer of the curable product or product component, to receive location information of the manufacturing site of the curable product or product component, and/or to receive determined feature information of the product or product component produced on basis of the sent recipe.

According to one embodiment, the system may comprise a machine learning model for determining said recipe. The system may further comprise means for: teaching the machine learning model on basis of the determined feature information of the product or product component produced on basis of the sent recipe.

According to one embodiment, the first user interface and/or communication interface may further be adapted to send an order request to at least one supplier of sidestream based raw material on basis of the determined recipe.

According to one embodiment, the order request may include location information of the manufacturing site of the curable product or product component.

According to one embodiment, the information related to the available sidestream based and/or virgin raw materials suitable for production of curable products comprises at least information on the amount, location and/or at least one feature of available side-stream based and/or virgin raw materials suitable for production of curable products.

According to one embodiment, the target feature information and/or the determined feature information of the curable product or product component may include at least one of the following: compressive strength, flexural tensile strength, splitting tensile strength, density, structural weight, operating conditions, CO2 emissions, natural resources consumption and/or price.

According to one embodiment, the device may comprise means for: receiving information related to available sidestream based and/or virgin raw materials suitable for production of curable products; sending target feature information related to sidestream based raw materials suitable for production of curable products to improve usability of at least one sidestream based raw material in production of the curable product or product component; and sending an order request to at least one supplier of sidestream based raw material.

According to one embodiment, the device may further comprise means for: sending an order request to at least one supplier of virgin raw material.

According to one embodiment, the device may further comprise: an interface and/or a communication interface for receiving information related to side-stream based and/or virgin raw materials suitable for production of curable products, and/or for sending an order request.

According to one embodiment, the information related to available sidestream based and/or virgin raw materials suitable for production of curable products may include information on the amount, location and/or at least one feature of available sidestream based and/or virgin raw materials suitable for production of curable products.

According to one embodiment, the device may further comprise means for delivering the information related to available sidestream based and/or virgin raw materials to a machine learning model adapted to determine a recipe for producing a curable product or product component on basis of the information related to available sidestream based and/or virgin raw materials and target feature information of the curable component or product component.

According to one embodiment, the device may further comprise means for: determining target feature information related to sidestream based raw materials suitable for production of curable products to improve usability of at least one sidestream based raw material in production of curable product of product component on basis of target feature information of the curable product or product component.

According to one embodiment, the device may further comprise means for: determining at least one additive for producing the curable product or product component on basis of the determined recipe.

According to one embodiment, the order request to at least one supplier of virgin raw material may include location information of the manufacturing site of the curable product or product component.

According to one embodiment, the order request to at least one supplier of virgin raw material may contain information on the determined at least one additive.

According to one embodiment, the order request to at least one supplier of sidestream based raw material may contain location information of the manufacturing site of the curable product or product component.

According to one embodiment, the target feature information of the curable product or product component may include at least one of the following: compressive strength, flexural tensile strength, splitting tensile strength, density, structural weight, operating conditions, CO2 emissions, natural resources consumption and/or price.

According to one embodiment, the device may comprise means for: receiving a request to deliver a recipe to be used in production of a curable product or product component, the request comprising target feature information of the curable product or product component; sending a recipe for producing the requested curable product or product component; and receiving determined feature information of the product or product component produced on basis of the sent recipe.

According to one embodiment, the device may further comprise: a user interface and/or communication interface for receiving a request to deliver a recipe of a curable product or product component, for sending the determined recipe for producing the requested curable product or product component, and/or for receiving a determined feature information of the product or product component produced on basis of the sent recipe.

According to one embodiment, the target feature information and/or the determined feature information of the curable product or product component may include feature information determined during manufacturing process of the product or product component, feature information determined during use of the product or product component, and/or feature information determined after use of the product or product component.

According to one embodiment, the feature information determined during use of the product or product component may include data measured by at least one sensor integrated in the product or product component or data derived from data measured by at least one sensor integrated in the product or product component.

According to one embodiment, the target feature information and/or the determined feature information of the curable product or product component includes at least one of the following: compressive strength, flexural tensile strength, splitting tensile strength, density, structural weight, operating conditions, CO2 emissions, natural resources consumption or price.

According to one embodiment, the device may further comprise means for: providing target feature information of the requested curable product or product component to the machine learning model which is adapted to produce a recipe for producing the requested curable product or product component on basis of target feature information of the curable product or product component and information related to available sidestream based and/or virgin raw materials; and/or receiving a recipe for producing the requested curable product or product component from a machine learning model.

According to one embodiment, the device may further comprise means for delivering determined feature information of the product or product component produced on basis of the sent recipe to the machine learning model for teaching the machine learning model.

According to one embodiment, the method may comprise: receiving information related to available sidestream based and/or virgin raw materials suitable for production of curable products; receiving a request to deliver a recipe of the curable product or product component, the request comprising target feature information of the curable product or product component; and determining a recipe for producing the requested curable product or product component on basis of target feature information of the requested curable product or product component and information related to available side-stream based and/or virgin raw materials

According to one embodiment, the method may comprise: receiving information related to available sidestream based and/or virgin raw materials suitable for production of curable products; sending target feature information related to sidestream based raw materials suitable for production of curable products to improve usability of at least one sidestream based raw material in production of a curable product or product component; and sending an order request to at least one supplier of sidestream based raw material.

According to one embodiment, the method may comprise: receiving a request to deliver a recipe to be used in production of a curable product or product component, the request comprising target feature information of the curable product or product component; sending a recipe for producing the requested curable product or product component; and receiving determined feature information of the product or product component produced on basis of the sent recipe

According to one embodiment, the computer program may comprise program code means for causing the device to perform any of the above-mentioned methods when said computer program is executed on the device.

Thus, the present disclosure relates to a system, devices, methods, and computer programs for producing a recipe for curable compositions.

### LIST OF FIGURES

Embodiments of the present description will be described in more detail below with reference to the accompanying Figures, in which:

FIG. 1 shows an example of a system for determining the recipe for curable compositions according to one embodiment;

FIG. 2 shows an example of a device which may be used to implement at least one embodiment according to one embodiment;

FIG. 3 shows an example of a neural network for determining the recipe for curable compositions according to one embodiment;

FIG. 4 shows an example of a neural network node for determining the recipe for curable compositions according to one embodiment;

FIG. 5 shows an example of a convolutional neural network for determining the recipe for curable compositions according to one embodiment;

FIG. 6 shows an example of communication and functionality for determining the recipe for curable compositions according to one embodiment;

FIG. 7 shows an example of a flow chart for determining a recipe for curable compositions and for transmitting it to the manufacturer of the end-product according to one embodiment;

FIG. 8 shows an example of a flow chart for improving the usability of sidestream based raw materials according to one embodiment; and

FIG. 9 shows an example of a flow chart for improving a recipe according to one embodiment.

In the Figures, the same reference numerals are used for the corresponding parts.

### DETAILED DESCRIPTION

In the present description, reference is now made to various embodiments, examples of which are shown in the Figures. The detailed description below, together with the Figures, is intended to illustrate the example in question and not to represent the only form in which the application illustrated by this example may be implemented. The description further provides exemplary functions and possible sequences of operations for implementing the illustrated embodiments. However, the same functionality may be achieved in other ways as well.

Utilizing the sidestreams of industrial processes offers opportunities to find new technological and environmental innovations. Curable compositions, for example geopolymer-based building materials, such as geopolymer elements may be produced from sidestreams of energy industry, mining industry, steel industry, and forest industry. Other applications for curable compositions include land building and stabilization, as well as filling and protection solutions for mining industry.

Different industrial processes produce a wide variety of sidestream raw materials, the amount, composition and availability (e.g., location or schedule) of which may vary considerably. Therefore, determining an optimal or suitable end-product from the available raw materials may be difficult.

According to one embodiment, the system may receive information related to available sidestream based and/or virgin raw materials suitable for production of curable compositions. The information on the composition of the substances may be measured, for example, with an XRF analyser (X-ray fluorescence). In addition, the system may receive a request to deliver a recipe for a curable end-product. The request may include target feature information of an end-product. In addition, the system may determine the recipe for the requested end-product on basis of the received target feature information and the information related to available raw materials. In addition, the system may provide separate user interface and/or communication interfaces for producers of raw materials and manufacturers of finished products. The system improves the usage of side-stream based materials in production of curable products or product components.

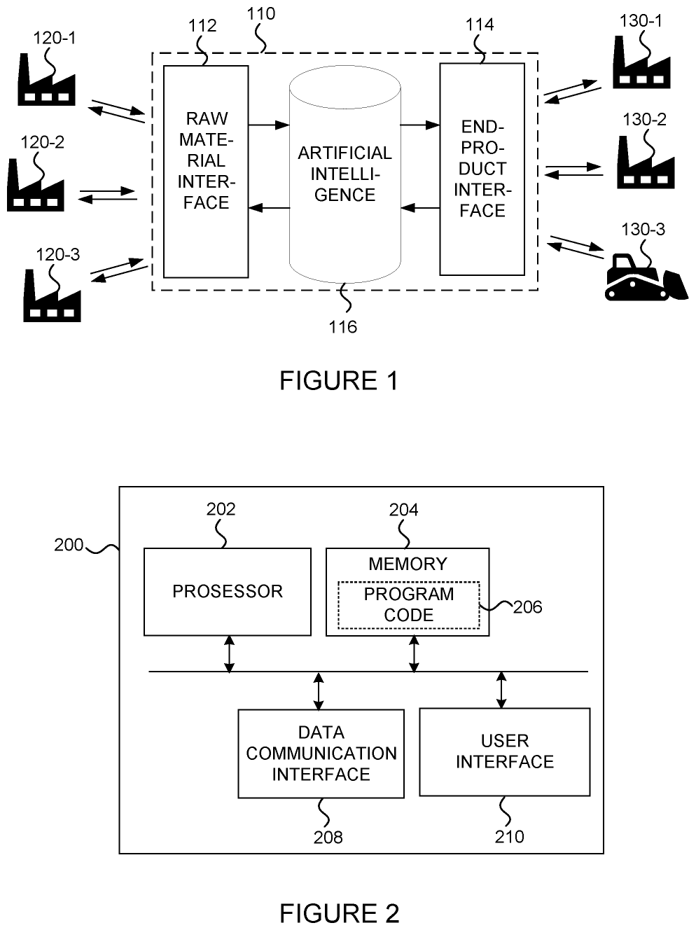

FIG. 1 shows an example of a system for determining the recipe for curable compositions according to one embodiment. The curable composition may be, for example, a geopolymer-based product, an alkali-activated material, a product curable by hydrotation reaction, or the like. The curable composition may be, for example, a slurry which, when dried, hardens. The curable composition may also be cured by incineration, which, in addition to drying, may produce favourable thermal effects in the composition. System 110 includes a raw material interface 112, an end-product interface 114, and an artificial intelligence model 116. Via the raw material interface 112, one or more raw material sources 120-1, 120-2, 120-3 may communicate with the system 110 to transmit information on available sidestream based and/or virgin materials. Sidestream based materials may include, for example, materials generated as sidestreams of industrial process. Virgin raw materials may contain materials which are not sidestream based. Examples of materials suitable for production of sidestream based curable products are coal-fired power plant ash, bio-ash, steel industry slag, green liquor sludge, waste incineration ash and slag, slag from hydrogen reduction steelmaking industry, tailings and side stones from mining industry, and neutralizing waste. Examples of virgin materials are natural stones or stone aggregates, sand, gravel, clays, silt, desert sands, and other acidic or alkaline soils, such as Latossolo-type soils.

Via the end-product interface 114, one or more end-product manufacturers 130-1, 130-2, 130-3 may communicate with the system 110 to transmit, for example, a request for a desired end-product and its target features, and to receive a recipe for producing the end-product. In addition, any end-product manufacturer 130 may provide feedback on the end-product produced on basis of the recipe. For example, the feedback may include information on the measured or otherwise determined features of the end-product. The end-product manufacturer 130 may be, for example, a geopolymer element plant, a civil engineering company, or another end user or dis-tributor of a curable product. The end-product may comprise a product or a product component.

The artificial intelligence model 116 may comprise, for example, a machine learning model, such as a neural network or other machine learning model. Alternatively, the artificial intelligence model 116 may be implemented by one or more algorithms. Based on the se-lected end-products with certain sets of raw materials, the artificial intelligence model 116 may be configured or taught to determine the optimal or suitable recipe for the requested end-product. The recipe may comprise, as one example, the amounts of necessary available raw materials or their ratios to produce at least one end-product. The recipe may further comprise instructions for preparation. Thus, the recipe may comprise, for example, one or more of the following: recipe, mixing order, mixing time, mixing conditions, mixing power, compaction method, compaction time, or information on indicative drying conditions. The artificial intelligence model 116 may also be re-trained or reconfigured based on feedback from the end-product manufacturer 130.

FIG. 2 shows an example of a device which may be used to implement at least one embodiment. Device 200 may have at least one processor 202. Although the device of FIG. 2 shows only one processor 202, the device 200 may include multiple processors. In one embodiment, processor 202 may be implemented as a multi-core processor, a single-core processor, or a combination of one or more single-core processors and/or one or more multi-core processors. For example, the processor 202 may be implemented as one or more different processing devices, such as an auxiliary processor, a mi-croprocessor, a controller, a digital signal processor (DSP), a processing circuit with or without a DSP, or various other processing devices, including an application specific integrated circuit (ASIC); field programmable gate array (FPGA) circuit, microcontroller unit, hardware accelerator, or the like. In one embodiment, the processor 202 may be configured to perform hard coded functionality. In one embodiment, the processor 202 may be implemented as an executor of software instructions, where the processor 202 may be configured with instructions to perform the functions described in this speci-fication when the instructions are run.

The device 200 may have at least one memory 204. Memory 204 may be implemented as one or more non-volatile memory devices, one or more non-volatile memory devices, and/or a combination of one or more non-volatile memory devices and one or more non-volatile memory devices. For example, the memory 204 may be implemented as a semiconductor memory, such as a PROM (programmable ROM) memory, an EPROM (erasable PROM) memory, a flash ROM, a RAM (random access memory), and so on.

The memory 204 may contain program code 206. The program code may be computer program code. In one embodiment, the program code 206 may include instructions, for example, for running the operating system and/or various applications. At least one memory 204 and program code 206 may be arranged with at least one processor 202 to cause the device 200 to operate in accordance with at least one embodiment when the program code 206 is executed by at least one processor 202.

The device 200 may have a communication interface 208 which allows the device 200 to send and receive information. The communication interface 208 may comprise at least one wireless radio connection, for example a third, fourth, fifth or later generation mobile connection, a wireless LAN connection, and/or a wired In-ternet connection. The communication interface 208 may further include specifications for the format of the information to be transferred, such as information related to raw materials or manufactured products. For example, the communication interface may include a pro-tocol for transmitting the necessary information. The communication interface may be implemented at least in part as a computer program. Device 200 may have or the device 200 may provide, via another device, a user interface 210. The user interface 210 may include, for example, a keyboard, display, touch screen, microphone, speaker, or integrated control buttons. The user interface may be arranged to transmit information, for example information related to raw materials or products to be manufactured, between the system and the system user. The various components of the device 200, such as the processor 202, the memory 204, the communication interface 208, and/or the user interface 210, may be arranged to communicate with each other over or via a communication link, such as a bus. The communication connection may be arranged, for example, on a printed circuit board, such as a motherboard or the like. The user interface may be implemented at least in part as a computer program.

The device 200 may implement the system of FIG. 1 or the device 200 may be part of the system of FIG. 1. The device 200 described and illustrated herein is merely an example of a device that may be utilized to implement the present embodiments and is not intended to limit the scope of the claims. It should be noted that the device 200 may include more or fewer components than shown in FIG. 2. For example, the device 200 may be distributed into several different physical entities which communicate with each other via a suitable communication connection. The operation of the device 200 may be implemented for example as a cloud service.

When the device 200 is arranged to perform a certain function, at least some of the components of the device, for example the processor 202 and/or the memory 204, may be arranged to perform this function. Further, when the processor 202 is arranged to perform a particular operation, it may be executed based on the program code 206.

The device 200 may include means for performing at least one of the methods described in the present description. These means may include, for example, at least one processor 202 and at least one memory 204 which includes program code 206. Memory 204 and program code 206, together with processor 202, may be configured to cause the device 200 to perform at least one of the methods shown. The device 200 may be, for example, a server or other computer.

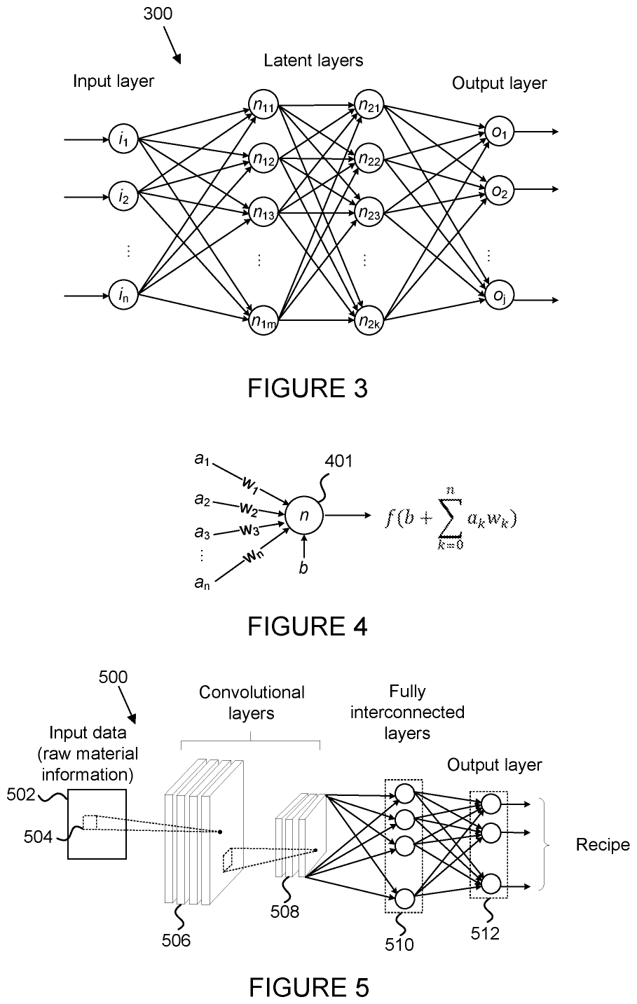

FIG. 3 shows an example of a neural network for determining the recipe for curable compositions according to one embodiment. The neural network is a computational model in which computation takes place in layers. The neural network 300 may include an input layer, one or more latent layers, and an output layer. Input layer nodes, i₁-iₙ may be connected to one or more of the nodes, n₁ₗ-n₁ₘ of the first latent layer. The nodes of the first latent layer may be connected to one or more nodes n₂ₗ-n₂ₖ of the second latent layer. Although the neural network of FIG. 3 comprises only two latent layers, it should be noted that the neural networks of different embodiments may have any number of latent layers.

The nodes of the last latent layer, as in the example of FIG. 3, the nodes n₂ₗ-n₂ₖ of the second latent layer may be connected to one or more output layer nodes, oₗ-oⱼ. It should be noted that there may be a different number of nodes in different layers. A node may also be called a neuron, a computational unit, or an elementary computational unit. The neural network is an example of a machine learning model, but the machine learning model may also be implemented in other ways. The neural network 300 may be taught to produce the desired recipe for producing curable products on basis of available sidestream based raw materials. For example, the input to the neural network 300 may include a vector indicative of the availability or amount of virgin and/or sidestream based raw materials known to the system. The neural network 300 may output a vector indicating relative proportions of the various raw materials in the end-product according to the determined recipe. The neural network 300 may be taught to implement a recipe determination task on basis of collected data, as described in more detail below.

FIG. 4 shows an example of a neural network node for determining the recipe for curable compositions according to one embodiment. The node 401 may have one or more inputs, a₁-aₙ, from one or more nodes of the preceding layer or other layer. Node 401 calculates the output value based on the input values. Inputs may be weighted by different coefficients w₁-wₙ. In this way, it is possible to adjust the effect of the output of each neural network node on the output of next node, and thus on the output of the entire neural network, for example on the recipe of the curable end-product. Input values a₁-aₙ may be multiplied by for example a coefficient w₁-wₙ associated with each input. Node 401 may further combine the input values into an output, for example, by calculating the sum of the weighted input values. The output of a node may also be referred to as activation. The node may also use a so-called bias value to add a constant b to the output. Weighting factors w₁-wₙ and bias value b are examples of neural network parameters to be taught. For example, when the neural network 300 is taught to determine a recipe, weighting factors and/or bias values may be updated until the neural network output (recipe) at a particular training input (raw data) is sufficiently close to the desired output.

In addition, the output of the node 401 may be controlled by an activation function f( ), which determines when and what kind of output the node 601 provides. Activation function f( ) may be, for example, a function that is substantially linear around zero, but limits the output value as the input increases or decreases. Examples of activation functions are the step function, the sigmoid function, the tanh function, the rectified linear unit (ReLu), and the softmax function. The output of node 401 may be transmitted to the nodes of one or more next and/or previous layers.

As noted above, neural networks may be taught using instructional data. The teaching algorithm may include changing the parameters of the neural network to achieve the desired output at a particular teaching input. For example, the neural network may be taught to produce a recipe on basis of raw materials available for producing curable products. By collecting enough data on the raw materials and the end-products that may be made from them that are suitable for their uses, the neural network may be taught to model and even to improve the process of manually searching for suitable end-products for a range of available raw materials.

During teaching, the output produced by the neural network may be compared with the desired, previously known data, e.g., ground-truth data. Ground-truth data may contain manually or otherwise determined recipes for end-products having the desired features. The difference between the output and the desired output may be modelled with an error function, which may also be called a loss function. Gradients for the taught neural network parameters may be calculated for the error function, and based on this, the neural network parameters may be updated to get closer to the desired output. This may be done, for example, by using a backpropagation algorithm in which gradients are specified layer by layer starting from the output layer until the parameters of the layers are updated. An example of an error function is the mean square error between the output and the desired output. Teaching is an iterative process in which the error or loss of the neural network is gradually reduced so that the neural network can produce the desired output also for input data which it has not been taught with.

FIG. 5 shows an example of a convolutional neural network for determining a recipe for curable compositions according to one embodiment. The convolutional neural network 500 includes at least one convolutional layer which may perform convolutional operations to separate or extract information from input data 502 and to produce feature maps 506. Input data 502 may include, for example, a matrix or a tensor which describes available raw materials. The raw materials may be sidestream based or virgin raw materials. The input data may also contain reservations for one or more additives which may be used to improve the end-product features associated with the recipe determined by the neural network. Thus, for example, the input data may be a matrix having rows which describe different materials and columns which describe certain features of each material. In general, the input may be a multidimensional tensor which describes features of the input raw materials (e.g., amount, location, composition, etc.). If a raw material is not available, the associated tensor elements may be initialized to a known value, for example zero. In this way, the neural network may be generalized to as many different combinations of raw materials as possible. The input data 502 may also include target feature information of the end-product according to the recipe. In this way, the neural network 500 can be taught to produce from available raw materials a recipe that best matches the target feature information. The target feature information may be included in the input data, for example, by adding vector elements, matrix columns, or generally by increasing the tensor dimensions.

The feature map may be generated by using a filter or kernel in a subset of the input data, e.g., input data block 504, and by sliding the filter through the input data 502 to obtain a value for each element of the feature map. The filter or core may be a matrix or tensor multiplied by a subset of the corresponding input data at each position. Multiple feature maps may be obtained by using multiple filters on the same input data. The next convolutional layer may take in the feature maps 506 produced by the previous layer and produce new feature maps 508. Filtering coefficients are teachable parameters and may be taught like the neural network 300. The convolutional network 500 may include one or more non-convolutional layers, for example one or more fully interconnected layers 510 before, after, or between the convolutional layers. The output layer 512 provides the output of the convolutional neural network 500, which after training contains a recipe for producing the curable end-product, as described above. With the machine learning model, the determination of the recipe may be automated, which allows the determination of the recipe for several different raw materials and end-products. In addition, a well-taught machine learning model is also capable of determining new, previously unknown end-products having the desired features.

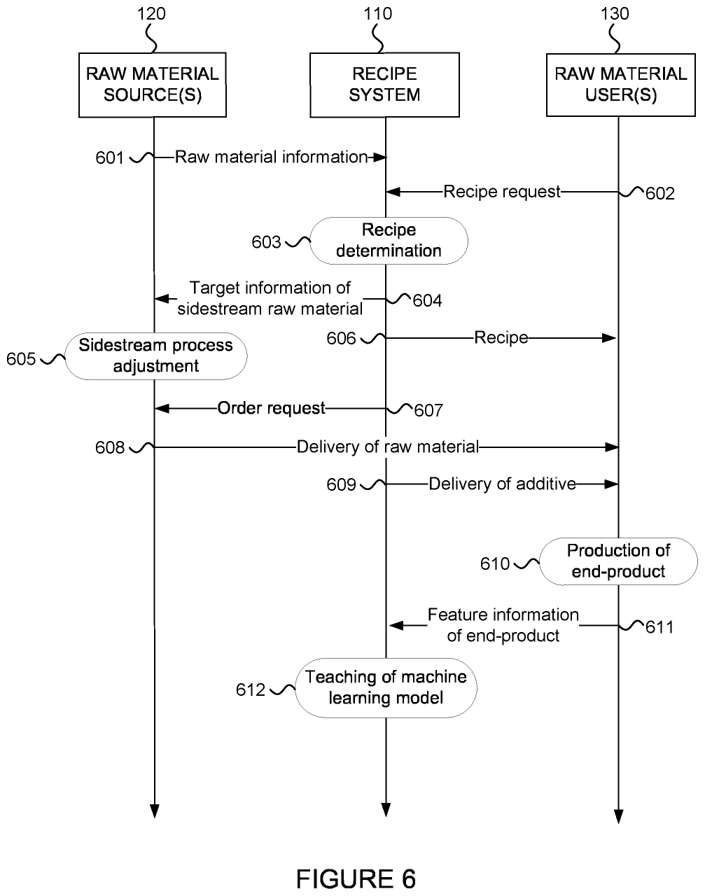

FIG. 6 shows an example of communication and functionality for determining a recipe for curable materials according to one embodiment.

At 601, one or more raw material sources 120 may transmit raw material information to the recipe system 110. Accordingly, the recipe system 110 may receive raw material information from one or more raw material sources 120 or other information sources, for example, a party representing the raw material source, such as a system or a user. The user may be a person. The raw material information may include information related to available sidestream based and/or virgin raw materials suitable for production of curable products. The raw material information may also include predictive information about the raw materials and/or their features. For example, a time point in the future at which the sidestream raw material becomes available may be specified for the sidestream process. It is also possible to evaluate, based on the sidestream process, for example one or more process parameters such as temperature, the features of the produced sidestream based raw material. Such prediction information may be included in the raw material information, allowing the determination of the recipe even before the sidestream based raw material is prepared. Thus, the available material may be an already existing material or a material which will be available only later.

The raw material information may be received via the first user interface and/or communication interface, for example the raw material interface 112. For example, the recipe system 110 may provide a web-based user interface for the user of the raw material source 120 for sending raw material information. Alternatively, the system integrated in the raw material source equipment may automatically or based on user input prepare a raw material report which is transmitted to the recipe system 110 via the first communication interface, e.g., as one or more messages. The raw material information may include at least information on amount, location and/or at least one feature of available sidestream based and/or virgin raw materials available for production of curable products.

At 602, one or more raw material users 130, for example manufacturer of the end-product, may transmit a recipe request to the recipe system 110. The recipe request may include a request to deliver a recipe of a curable product or product component. The requested product, products or product component may also be referred to as the end-product. The recipe request may contain target information of the end-product, for example, target feature information of the curable product or product component. Accordingly, the recipe system 110 may receive a recipe request from one or more raw material users 130 or another source of information, such as a representative of the raw material user, such as a system or user.

The recipe request may be received via another user interface and/or communication interface, for example the end-product interface 114. For example, the recipe system may provide a web-based user interface for the raw material user 130 for sending a recipe request. Alternatively, the system integrated into the raw material user's equipment may automatically, or based on user input, prepare a recipe request which is transmitted to the recipe system 110 via another communication interface, for example, as one or more messages.

The target information of the curable product or product component according to the recipe request may include at least one of the following: compressive strength, flexural tensile strength, splitting tensile strength, density, structural weight, operating conditions, CO₂ emissions, natural resources consumption and/or price. Operating conditions may include information on, for example, whether the end-product is intended for outdoor or indoor use, or type of weather conditions it is exposed to. The natural resources consumption may be determined based on the mass of the materials according to the recipe (in units e.g., kg or t), but in any case, so that for sidestream based substances the natural resources consumption is zero. The price of the target product may be determined, for example, based on the composition according to the determined recipe and price information of the various raw materials. At least some of the raw material price information may be determined or estimated beforehand. At least some the price information may be received through the raw material interface from at least one raw material source 120, for example, as part of the raw material information at 601.

One or more raw material users 130 may further transmit information on available raw materials or location of the manufacturing site. Accordingly, the recipe system 110 may receive from one or more raw material users 130, information on raw materials available for the manufacturer of the curable product or product component or location information of the manufacturing site of the curable product or product component. These may be considered when determining the recipe for the requested product.

At 603, the recipe system 110 may determine a recipe for producing the requested curable product or product component. The recipe may be determined based on artificial intelligence 116, such as a machine learning model or an algorithm. The recipe system 110 may either include a machine learning model or communicate with an external machine learning model. The artificial intelligence 116 may determine the recipe based on the target feature information of the requested curable product or product component and the information related to the available sidestream based and/or virgin raw materials.

As noted above, the determined recipe may also include manufacturing instructions for producing the end-product. For example, the recipe system may be configured with parameters related to the manufacturing process for different raw materials. Manufacturing instructions may also be generated using a machine learning model. For example, the teaching data used for teaching the machine learning model may include, in addition to the raw materials and suitable end-products specified manually (or otherwise without machine learning), information on the parameters associated with the manufacturing process of these end-products. Parameters related to the manufacturing process may include, for example, information on mixing order of particular materials, mixing time, mixing conditions (e.g., temperature), mixing performance, compaction method, compaction duration, or recommended drying conditions. For a machine learning model, these known manufacturing process parameters may be entered as part of the teaching data, i.e., as input data when the machine learning model is taught.

According to one embodiment, the recipe system 110 may provide information related to available side-stream based and/or virgin raw materials to a machine learning model adapted to determine a recipe for producing a curable product or product component on basis of information related to available sidestream based and/or virgin raw material and target feature information of the curable product or product component. The machine learning model may be part of the recipe system 110. The machine learning model may also be located in another device or system, whereby the recipe system 110 may send a request to determine a recipe according to the end-product target feature information on basis of the raw material information. As discussed above, the machine learning model may be taught to produce the requested recipe using, for example, manually generated training data. The recipe system 110 may receive a recipe for producing a desired curable product or product component from a machine learning model.

At least a portion of the raw materials in the determined recipe may include side-stream based and/or virgin raw materials according to the information received via the first user interface and/or communication interface, for example the raw material interface 112. The determined recipe may further include at least one raw material available to the raw material user 130, which may be a sidestream based or virgin raw material.

According to one embodiment, the determined recipe may include at least one additive. One or more additives may be configured in the recipe system 110. The additive may be a raw material which may be used in addition to sidestream based raw materials in production of the curable end-products, for example to improve their properties. The additive may be one of the sidestream based raw materials. The additive may be a reactive substance which acts on the efficiency of the reactions occurring in the manufacturing process. The additive may thus act as an enhancer or a compounding agent. The additive may be a chemical or other substance, for example silicon or aluminium. If, for example, the quantity of a particular reactive agent in the sidestream based raw material is too low to make the manufacturing process optimal or efficient enough, the concentration of this reactant may be increased by adding this reactive agent as an additive to improve the mixture ratio. The additive may be, for example, a plasticizer, a porosifier, a sealant, an accelerator, a retardant, or a colorant. Additives may be used to improve, for example, the strength, tightness and/or weather resistance of the end-product.

The supplier or operator of the recipe system 110 may for example have in storage one or more additives suitable for this purpose. As described above, these available additives may be considered when determining the recipe. The determined recipe may contain one or more additives. The additives included in the recipe may form a subset of possible additives.

According to one embodiment, the recipe system 110 may determine target feature information related to sidestream based raw materials suitable for production of curable products to improve usability of at least one sidestream based raw material in production of a curable product or product component on basis of target feature information of the curable product or product component. For example, the recipe system 110 may determine multiple recipes for producing the end-product on basis of raw materials according to raw material information and those which are slightly different from the raw material information. If the end-product according to the request can only be obtained with raw materials which differ from the raw material information, or for some reason is more advantageous to manufacture in that manner, the recipe system 110 may determine target feature information for sidestream based materials.

The usability of the sidestream based raw material may be improved, for example, to maintain its reactivity. This target feature information may include a modification in at least one feature of one or more available sidestream based raw materials. This allows the sidestream process to be optimized or improved to produce the requested end-product. The sidestream process may be optimized, for example, by modifying the sidestream cooling, grinding (particle size) or heating. The sidestream process may be optimized mechanically, thermally, or chemically.

At 604, the recipe system 110 may send target feature information related to sidestream based raw materials suitable for production of curable products to at least one raw material source 120, for example via a first user interface and/or communication interface (raw material interface 112). Correspondingly, the raw material source 120 may receive the target feature information of this sidestream raw material.

At 605, the raw material source 120 may adjust the sidestream process so that the features of at least one sidestream based raw material correspond or approach its target features. Contrary to FIG. 6, it is possible that at this stage the raw material source sends to the recipe system 110 information on post-adjustment side-stream raw material (cf. raw material information 601), and that the recipe system determines a new recipe on basis of the updated raw material information (cf. 603). The sidestream process adjustment 605 allows the properties of the sidestream based raw materials to be improved to produce the requested end-product, and thus to improve the properties of the end-product according to the determined recipe. The sidestream process adjustment may include, for example, a change in the combustion temperature of the sidestream process, for example to reduce residual carbon.

At 606, the recipe system 110 may send the determined recipe for producing the requested curable product or product component. The recipe may be sent to the raw material user 130. The recipe may be sent via another user interface and/or communication interface, for example the end-product interface 114. The recipe may be sent, for example, as a reply message to the recipe request of 602.

At 607, the recipe system 110 may send an order request to at least one supplier of sidestream based and/or virgin raw material. The order request may be sent on basis of the determined recipe. The recipe may be sent via a first user interface and/or communication interface, for example the raw material interface 112. The order request may include a request to deliver at least one sidestream based and/or virgin material. The order request may contain the identifier of at least one material to be ordered. The order request may further include information related to the delivery of the order, such as location information of the raw material user 130 or the manufacturer of the end-product or the manufacturing site of the end-product. In addition, the order request may contain other information, such as information about the desired or required delivery time. According to one embodiment, the order request may also include target feature information of the sidestream based raw material, as in 604. In this case, the raw material source 120 may adjust its sidestream process based on the order request, correspondingly as at 605. Correspondingly, an order request may also be sent to at least one supplier of the determined additive.

At 608, the raw material source 120 may deliver the raw material in accordance with the order request to the raw material user 130. It should be noted that the recipe system 110 allows the delivery of raw materials according to the determined recipe directly from raw material sources 120 to raw material users 130, which reduces the logistical costs of raw materials suitable for production of sidestream based and/or virgin material based curable products.

At 609, the supplier or operator of the recipe system 110 may supply at least one additive according to the determined recipe to at least one user of the raw material 130.

At 610, the raw material user 130 may produce the end-product on basis of the determined recipe. The recipe may contain at least one secondary raw material supplied by the raw material sources 120. The recipe may further comprise at least one virgin raw material supplied by the raw material sources 120 or an additive supplied by the supplier or operator of the recipe system 110.

At 611, the user of the raw material may send, on basis of the recipe received at 606, determined feature information of the product or product component produced at 610. Correspondingly, the recipe system may receive determined feature information of the product or product component produced on basis of the recipe sent at 606. This feature information may be used to improve recipe determination, for example, by teaching artificial intelligence 116. Feature information of the end-product may be received via another user interface and/or communication interface, for example the end-product interface 114.

The raw material user 130 may determine the feature information of the finished end-product, for example, by measurements, calculations, or based on sensors integrated in the end-product. The determined feature information of the end-product may include feature information determined during manufacturing process of the curable product or product component, feature information determined during use, and/or feature information determined after use. An example of the feature information of the end-product is the moisture content of the pulp during manufacturing process, for example before or after incineration. Feedback information on the properties of the end-product during the manufacturing process allows the recipe to be optimized during the manufacturing process. The feature information of the end-product may also include information about the manufacturing process of the end-product, for example the temperature of the boiler. The feature information determined during use of the end-product allows to monitor the features of the end-product and to improve the determination of the recipe for future manufacturing processes. Some of the features of the end-product may not necessarily be determined during use. Therefore, it may be advantageous to receive feedback on the features determined after use of the end-product. This will allow improving of the recipe determination for future manufacturing processes.

According to one embodiment, the feature information determined during use of the product or product component may include data measured by at least one sensor integrated in the product or product component. The data measured by the sensor may be received via another communication interface, for example the end-product interface 114. In addition, or alternatively, the feature information determined during use may include data derived from data measured by at least one sensor integrated in the product or product component. This data may be derived in the system of the raw material user 130 automatically, for example, by calculating certain feature information based on the data produced by one or more sensors. The feature information may also be derived or measured manually, allowing the user to transmit the measured or derived information to the recipe system 110, for example, via a second user interface. The determined feature information of the end-product may include at least one of the following: compressive strength, flexural tensile strength, splitting tensile strength, density, structural weight, operating conditions, CO2 emissions, natural resources consumption or price. Price of the end-product is an example of a determined feature which cannot be directly measured. However, it may be determined, for example, based on the resources used for the end-product composition and in the manufacture thereof, such as electricity consumption. The feature information determined during use may also include information determined or measured during installation of the end-product, for example information on an impact event applied on a junction pile made of the curable composition. The feature information during use of the end-product may include information on the deflection, tilting, vibration, salinity and/or water level of the ground surrounding the end-product, such as the junction pile in question. This allows the product to be monitored throughout its life cycle, and the data collected may be further used to improve recipe determination.

At 612, the recipe system 110 may teach the machine learning model on basis of the determined feature information of the product or product component produced on basis of the sent recipe. For example, the recipe system 110 may provide determined feature information of the manufactured product or product component to the machine learning model to teach the machine learning model. Teaching may include retraining or further teaching of the machine learning model. At this stage, the training may be based, for example, on another error function calculated, for example, from the target feature information received at 602 and the determined feature information received at 611. The second error function may for example include the difference between the target feature information and the feature information or its absolute value. For example, the target feature information and the feature information may be given as vectors in which a particular element numerically describes a particular feature. The second error function may include, for example, a norm for the separation of these vectors, for example a Euclidean norm. The same teaching data as before may be used for re-teaching or further teaching, but with the help of another error function, the machine learning model may be taught to consider the received feedback on the functionality of the recipe.

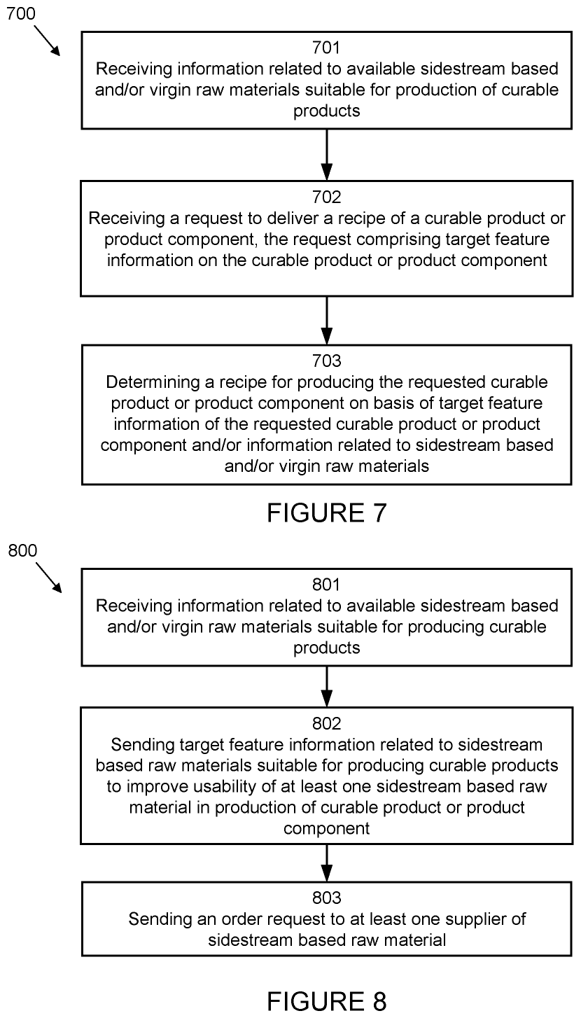

FIG. 7 shows an example of a flow chart for determining the recipe for curable compositions and for transmitting it to the end-product manufacturer according to one embodiment.

At 701, information related to available side-stream based and/or virgin raw materials suitable for production of curable products is received.

At 702, a request to deliver a recipe for the curable product or product component is received, the request comprising target feature information of the curable product or product component.

At 703 is determined a recipe for producing the requested curable product or product component on basis of target feature information of the requested curable product or product component and information related to available sidestream based and/or virgin raw materials.

FIG. 8 shows an example of a flow chart for a method for improving the usability of sidestream based raw materials according to one embodiment.

At 801, information related to available side-stream based and/or virgin raw materials suitable for production of curable products is received.

At 802, target feature information related to sidestream based raw materials suitable for production of curable products is sent to improve usability of at least one sidestream based raw material in production of a curable product or product component.

At 803, an order request is sent to at least one supplier of sidestream based raw material.

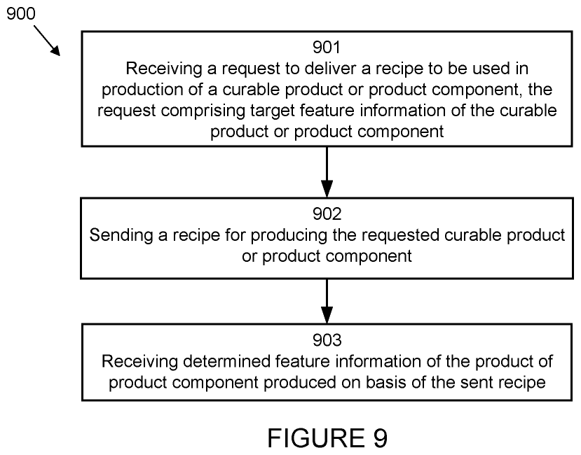

FIG. 9 shows an example of a flow chart for a method for improving a recipe according to one embodiment.

At 901, a request to provide a recipe to be used in production of a curable product or product component is received, the request comprising target feature information of the curable product or product component.

At 902, a recipe for producing the requested curable product or product component is sent.

At 903, determined feature information of the product or product component produced on basis of the sent recipe is received.

Other embodiments of the methods are based directly on the operation of the disclosed devices and systems, as set forth in the claims, application text, and figures, and are therefore not repeated herein.

The device may be arranged to perform any method or function according to the present description. The computer program or computer program product may include instructions that cause the device to perform any method or function as described herein when the instructions are run. The device or system may have means for performing any method or function as described herein.

The steps or functions of the disclosed embodiments may be performed in any suitable order, or partially or completely simultaneously. Also, the various embodiments may not include all of the structures, features, or functions shown. In addition, any embodiment may be combined with one or more other embodiments, un-less this possibility is specifically denied.

The above advantages may be associated with one embodiment or may be associated with more than one embodiment. Embodiments are not limited to solutions that solve one or more of said problems or have one or more of said advantages. When structures, features, or functions have been discussed in a unit, they may potentially be applied to many similar units, and vice versa.

The terms ‘including’ and ‘comprising’ mean that the methods and devices disclosed may include said features, but that said features do not constitute an exhaustive list of features of the method or the device. Thus, the methods or devices disclosed may include other features.

The above embodiments are not to be construed as limiting the scope of the requirements set forth below, but the basic idea may be modified in many ways without departing from the scope of the requirements.

1. A system, comprising:
at least one processor; and
at least one memory including program code, the at least one memory and the program code configured to, with the at least one processor, cause the system to:
receive information related to at least one of available sidestream based or virgin raw materials suitable for production of curable products via at least one of a first user interface or data communication interface;
receive a request for delivering a recipe for a curable product or product component via at least one of a second user interface or communication interface, the request comprising target feature information of the curable product or product component;
determine a recipe for producing the requested curable product or product component on basis of the target feature information of the curable product or product component and the information related to at least one of available sidestream based or virgin raw materials; and
send the determined recipe for producing the requested curable product or product component via at least one of the second user interface or communication interface.

2. The system according to claim 1, wherein at least a portion of the raw materials contained in the determined recipe includes at least one of sidestream based or virgin raw materials in accordance with the information received through the at least one of the first user interface or communication interface.

3. The system according to claim 1, wherein the at least one memory and the program code are further configured to, with the at least one processor, cause the system to:
determine target feature information related to sidestream based raw materials suitable for production of curable products to improve usability of at least one sidestream based raw material in production of the curable product or product component on basis of the target feature information of the curable product or product component, wherein the at least one of the first user interface or communication interface is adapted to send the target feature information related to the sidestream based raw materials suitable for production of curable products.

4. The system according to claim 1, wherein the at least one of the second user interface or communication interface is further adapted to receive at least one of information on raw materials available from a manufacturer of the curable product or product component, location information of a manufacturing site of the curable product or product component, or determined feature information of the product or product component produced on basis of the sent recipe.

5. The system according to claim 1, comprising a machine learning model configured to determine said recipe, wherein the at least one memory and the program code are further configured to, with the at least one processor, cause the system to:
teach the machine learning model on basis of the determined feature information of the product or product component produced on basis of the sent recipe.

6. The system according to claim 1, wherein the at least one of the first user interface or communication interface is further adapted to send an order request to at least one supplier of sidestream based raw material on basis of the determined recipe.

7. The system according to claim 4, wherein the order request includes location information of the manufacturing site of the curable product or product component.

8. The system according to claim 1, wherein the information related to at least one of available sidestream based or virgin raw materials suitable for production of curable products comprise at least information on at least one of amount, location or at least one feature of at least one of available sidestream based or virgin raw materials suitable for production of curable products.

9. The system according to claim 1, wherein at least one of the target feature information or the determined feature information of the curable product or product component includes at least one of the following: compressive strength, flexural tensile strength, splitting tensile strength, density, structural weight, operating conditions, CO2 emissions, natural resources consumption or price.

10. A device, comprising:
at least one processor; and
at least one memory including program code, the at least one memory and the program code configured to, with the at least one processor, cause the device to:
receive information related to at least one of available sidestream based or virgin raw materials suitable for production of curable products via at least one of a user interface or communication interface;
send target feature information related to sidestream based raw materials suitable for production of curable products to improve usability of at least one sidestream based raw material in production of a curable product or product component via the at least one of the user interface or communication interface; and
send an order request to at least one supplier of sidestream based raw material via the at least one of the user interface or communication interface.

11. The device according to claim 10, wherein the at least one memory and the program code are further configured to, with the at least one processor, cause the device to:
send an order request to at least one supplier of virgin raw material.

12. The device according to claim 10, wherein the information related to the at least one of available sidestream based or virgin raw materials suitable for production of curable products includes information on at least one of amount, location or at least one feature of the at least one of available sidestream based or virgin raw materials suitable for production of curable products.

13. The device according to claim 10, wherein the at least one memory and the program code are further configured to, with the at least one processor, cause the device to:
provide the information related to the at least one of available sidestream based or virgin raw materials to a machine learning model adapted to determine a recipe for producing the curable product or product component on basis of information on the at least one of available sidestream based or virgin raw materials and the target feature information of the curable product or product component.

14. The device according to claim 13, wherein the at least one memory and the program code are further configured to, with the at least one processor, cause the device to:
determine target feature information related to sidestream based raw materials suitable for production of curable products to improve usability of at least one sidestream based raw material in production of the curable product or product component on basis of target feature information of the curable product or product component.

15. The device according to claim 10, wherein the at least one memory and the program code are further configured to, with the at least one processor, cause the device to: determine at least one additive for producing the curable product or product component based on the determined recipe.

16. The device according to claim 10, wherein the order request to at least one supplier of virgin raw material includes location information of a manufacturing site of the curable product or product component.

17. The device according to claim 15, wherein the order request to at least one supplier of virgin raw material includes information on the determined at least one additive.

18. The device according to claim 10, wherein the order request to at least one supplier of sidestream based raw material includes location information of a manufacturing site of the curable product or product component.

19. The device according to claim 10, wherein the target feature information of the curable product or product component includes at least one of the following:
compressive strength, flexural tensile strength, tensile strength, density, structural weight, operating conditions, CO2 emissions, natural resources consumption or price.

20. A device, comprising:
at least one processor; and
at least one memory including program code, the at least one memory and the program code configured to, with the at least one processor, cause the device to:
receive a request to deliver a recipe to be used in production of a curable product or product component via at least one of a user interface or communication interface, the request comprising target feature information of the curable product or product component;
send a recipe for producing the requested curable product or product component via the at least one of the user interface or communication interface; and
receive determined feature information of the product or product component produced on basis of the sent recipe via the at least one of the user interface or communication interface.

21. The device according to claim 20, wherein at least one of the target feature information or the determined feature information of the curable product or product component includes feature information determined during manufacturing process of the product or product component, feature information determined during use of at least one of the product or product component, or feature information determined after use of the product or product component.

22. The device according to claim 21, wherein the feature information determined during use of the product or product component includes data measured by at least one sensor integrated in the product or product component or data derived based on data measured by at least one sensor integrated in the product or product component.

23. The device according to claim 20, wherein at least one of the target feature information of the curable product or product component and/or the determined feature information includes at least one of the following: compressive strength, flexural tensile strength, splitting tensile strength, density, structural weight, operating conditions, CO2 emissions, natural resources consumption or price.

24. The device according to claim 20, wherein the at least one memory and the program code are further configured to, with the at least one processor, cause the device to:
provide the target feature information of the requested curable product or component to a machine learning model adapted to produce the recipe for producing the requested curable product or product component on basis of the target feature information of the curable product or product component and information related to at least one of available sidestream based or virgin raw materials; and
receive the recipe for producing the requested curable product or product component from the machine learning model.

25. The device according to claim 24, wherein the at least one memory and the program code are further configured to, with the at least one processor, cause the device to:
provide determined feature information of the product or product component produced on basis of the sent recipe to the machine learning model for teaching the machine learning model.

26. A method, comprising:
receiving by a device information related to at least one of available sidestream based or virgin raw materials suitable for production of curable products via at least one of a first user interface or communication interface;
receiving by the device a request to deliver a recipe of a curable product or product component, the request comprising target feature information of the curable product or product component via at least one of a second user interface or communication interface;
determining by the device a recipe for producing the requested curable product or product component based on target feature information of the requested curable product or product component and information related to the at least one of available sidestream based or virgin raw materials; and
sending the recipe determined by the device for producing the requested curable product or product component via the at least one of the second user interface or the communication interface.

27. A method, comprising:
receiving by a device information related to at least one of available sidestream based or virgin raw materials suitable for production of curable products via at least one of a user interface or the communication interface;
sending by the device target feature information related to sidestream based raw materials suitable for production of curable products to improve usability of at least one sidestream based raw material in production of the curable product or product component via the at least one of the user interface or the communication interface; and
sending by the device an order request to at least one supplier of sidestream based raw material via the at least one of the user interface or the communication interface.

28. A method comprising:
receiving by a device a request to deliver a recipe to be used in production of a curable product or product component via at least one of a user interface or communication interface, the request comprising target feature information of the curable product or product component;
sending by the device a recipe for producing the requested curable product or product component via the at least one of the user interface or the communication interface; and
receiving the determined feature information of the product or product component produced based on the recipe sent by the device via the at least one of the user interface and/or the communication interface.

29. A non-transitory computer-readable medium having instructions stored thereon that are executable by a processor, the instructions comprising:
instructions to receive by a device information related to at least one of available sidestream based or virgin raw materials suitable for production of curable products via at least one of a first user interface or communication interface:
instructions to receive by the device a request to deliver a recipe of a curable product or product component, the request comprising target feature information of the curable product or product component via at least one of a second user interface or communication interface;
instructions to determine by the device a recipe for producing the requested curable product or product component based on target feature information of the requested curable product or product component and information related to at least one of available sidestream based or virgin raw materials; and
instructions to send the recipe determined by the device for producing the requested curable product or product component via the at least one of the second user interface or the communication interface.

30. A non-transitory computer-readable medium having instructions stored thereon that are executable by a processor, the instructions comprising:
instructions to receive by a device information related to at least one of available sidestream based or virgin raw materials suitable for production of curable products via at least one of a user interface or the communication interface;
instructions to send by the device target feature information related to sidestream based raw materials suitable for production of curable products to improve usability of at least one sidestream based raw material in production of the curable product or product component via the at least one of the user interface or the communication interface; and
instructions to send by the device an order request to at least one supplier of sidestream based raw material via the at least one of the user interface or the communication interface.

31. A non-transitory computer-readable medium having instructions stored thereon that are executable by a processor, the instructions comprising:
instructions to receive by a device a request to deliver a recipe to be used in production of a curable product or product component via at least one of a user interface or communication interface, the request comprising target feature information of the curable product or product component;
instructions to send by the device a recipe for producing the requested curable product or product component via the at least one of the user interface or the communication interface; and
instructions to receive the determined feature information of the product or product component produced based on the recipe sent by the device via the at least one of the user interface or the communication interface.
